(19) United States
(12) Patent Application Publication
(10) Pub. No.: US 2019/0051407 A1
(43) Pub. Date: Feb. 14, 2019

(54) ASSET MANAGEMENT AND LOCATION SYSTEM

(71) Applicant: Nesa Solutions, Inc., Miami, FL (US)

(72) Inventors: Helmer Antonius Jacobus VAN DER VEER, Miami, FL (US); Neil SALEM, Pinecrest, FL (US)

(21) Appl. No.: 16/059,825

(22) Filed: Aug. 9, 2018

Related U.S. Application Data
(60) Provisional application No. 62/544,365, filed on Aug. 11, 2017.

(51) Int. Cl. G16H 40/40; G16H 40/63

(52) U.S. Cl. CPC G16H 40/40; G16H 40/63

(57) ABSTRACT

A management device for managing at least one item associated with a physical cart at a premises is provided. The management device includes processing circuitry where the processing circuitry includes a memory and a processor. The processing circuitry is configured to: receive cart data associated with the at least one item associated with the physical cart at the premises; determine whether to trigger application of at least one rule to the cart data; when triggered, apply the at least one rule to cart data based on the determination whether to trigger application of at least one rule; and perform at least one action based on the applied at least one rule.

### CROSS-REFERENCE TO RELATED APPLICATION

This Application is related to and claims priority to U.S. Provisional Patent Application No. 62/544,365, entitled “ASSET MANAGEMENT AND LOCATION SYSTEM,” filed Aug. 11, 2017, the entirety of which is incorporated herein by reference.

### TECHNICAL FIELD

This invention relates to asset management, and in particular to automated compliance and inventory management of assets associated with storage devices.

### INTRODUCTION

Inventory management in certain environments has long been a challenging problem. One such environment is a hospital or healthcare setting where certain items are used more often than other items, and where having certain items immediately available could make a difference in saving a patient's life. To further complicate inventory management in the healthcare setting, certain equipment such as defibrillators, suction pumps, etc., are stored in mobile carts, i.e., “crash carts”, that are used to transport the equipment and/or dispense medication at the site of a medical/surgical emergency. In other words, not only does the respective inventory of each cart change often but so can the location of these carts, thereby making inventorying of carts and items in these carts and/or in certain locations of a hospital, e.g., emergency room, difficult to track, discover, look up, and update.

### SUMMARY

According to one aspect of the disclosure, a management device for managing at least one item associated with a physical cart at a premises is provided. The management device includes processing circuitry where the processing circuitry includes a memory and a processor. The processing circuitry is configured to: receive cart data associated with the physical cart at the premises, determine whether to trigger application of at least one rule to the cart data, when triggered, apply the at least one rule to cart data based on the determination whether to trigger application of at least one rule, and perform at least one action based on the applied at least one rule.

According to one embodiment of this aspect, the at least one rule applied to the cart data is based on a location of the physical cart at the premises. According to one embodiment of this aspect, the premises includes a plurality of zones, each of the plurality of zones corresponding to a respective set of rules, each rule defining at least one characteristic of the at least one item. According to one embodiment of this aspect, the determining whether to trigger application of at least one rule to the cart data includes: determining whether the physical cart has moved to from a first zone to a second zone. The application of the at least one rule to the cart data is based on the determination that the physical cart has moved from the first zone to the second zone, the plurality of zones including the first and second zones.

According to one embodiment of this aspect, the processing circuitry is further configured to receive a notification of an alert at the premises where the at least one rule applied to the cart data corresponds to a predefined set of rules associated with the alert at the premises. According to one embodiment of this aspect, the processing circuitry is further configured to determine whether the cart data meets the at least one rule. The performing of the at least one action is based on the determination that the cart data does not meet the at least one rule, the at least one action including: searching the premises for at least one other physical cart, and if at least one other physical cart is found, interrogating the at least one other physical cart to determine whether the at least one other physical cart meets the at least one rule. According to one embodiment of this aspect, the at least one rule is taken from a group consisting of: a location of the cart and a notification of a change in a predefined state.

According to one embodiment of this aspect, the cart data includes at least one tag identifier associated with at least one item associated with the cart. According to one embodiment of this aspect, the at least one action includes at least one taken from a group consisting of: generating a notification, automatically ordering at least one item, triggering a visual alert on a display associated with the cart and triggering an interrogation of at least one other cart. According to one embodiment of this aspect, the processing circuitry is configured to provide an interface for modifying a virtual cart associated with the physical cart, the virtual cart defining at least one minimum characteristic for the at least one item.

According to another aspect of the disclosure, a method for a management device for managing at least one item associated with a physical cart at a premises. Cart data associated with the physical cart at the premises is received. A determination is made whether to trigger application of at least one rule to the cart data. When triggered, the at least one rule is applied to cart data based on the determination whether to trigger application of at least one rule. At least one action is performed based on the applied at least one rule.

According to one embodiment of this aspect, the at least one rule applied to the cart data is based on a location of the physical cart at the premises. According to one embodiment of this aspect, the premises includes a plurality of zones, each of the plurality of zones corresponding to a respective set of rules, each rule defining at least one characteristic of the at least one item. According to one embodiment of this aspect, the determining whether to trigger application of at least one rule to the cart data includes: determining whether the physical cart has moved to from a first zone to a second zone. The application of the at least one rule to the cart data is based on the determination that the physical cart has moved from the first zone to the second zone, the plurality of zones including the first and second zones.

According to one embodiment of this aspect, receiving a notification of an alert at the premises is received where the at least one rule applied to the cart data corresponds to a predefined set of rules associated with the alert at the premises. According to one embodiment of this aspect, a determination is made whether the cart data meets the at least one rule. The performing of the at least one action is based on the determination that the cart data does not meet the at least one rule, the at least one action including: searching the premises for at least one other physical cart; and if at least one other physical cart is found, interrogating the at least one other physical cart to determine whether the at least one other physical cart meets the at least one rule. According to one embodiment of this aspect, the at least one rule is taken from a group consisting of: a location of the cart and a notification of a change in a predefined state.

According to one embodiment of this aspect, the cart data includes at least one tag identifier associated with at least one item associated with the cart. According to one embodiment of this aspect, the at least one action includes at least one taken from a group consisting of: generating a notification, automatically ordering at least one item, triggering a visual alert on a display associated with the cart and triggering an interrogation of at least one other cart. According to one embodiment of this aspect, providing an interface for modifying a virtual cart associated with the physical cart is provided where the virtual cart defines at least one minimum characteristic for the at least one item.

According to another aspect of the disclosure, a method for a management device for managing at least one item associated with a physical cart at a premises is provided. The premises includes a plurality of zones including a first zone and a second zone where each of the plurality of zones corresponding to a respective set of rules and each rule defining at least one characteristic of at least one item. Cart data associated with the physical cart at the premises is received. A determination is made whether to trigger application of at least one rule to the cart data where the determining whether to trigger application of at least one rule to the cart data includes determining whether the physical cart has moved to from the first zone to the second zone. When triggered, the at least one rule is applied to cart data based on the determination whether to trigger application of at least one rule. A determination is made whether the cart data meets the at least one rule. At least one action is performed based on the applied at least one rule, the performing of the at least one action including: if the cart data does not meet the at least one rule, searching the premises for at least one other physical cart, and if at least one other physical cart is found, interrogating the at least one other physical cart to determine whether the at least one other physical cart meets the at least one rule, and generating a notification if the at least one other physical cart meets the at least one rule.

### BRIEF DESCRIPTION OF THE DRAWINGS

A more complete understanding of embodiments described herein, and the attendant advantages and features thereof, will be more readily understood by reference to the following detailed description when considered in conjunction with the accompanying drawings wherein:

FIG. 1 is a block diagram of an example system of for managing inventory in accordance with the principles of the invention;

FIG. 2 is a diagram of an example implementation of the system in accordance with the principles of the invention;

FIG. 3 is a flow diagram of an example virtualization process in accordance with the principles of the invention;

FIG. 4 is a flow diagram of an example management process in accordance with the principles of the invention;

FIG. 5 is a flow diagram of an example asset management process in accordance with the principles of the invention;

FIG. 6 is a block diagram of an alternative management device in accordance with the principles of the invention; and

FIG. 7 is an example interactive interface for modifying one or more virtual cart configurations in accordance with the principles of the invention.

### DETAILED DESCRIPTION

The contents of carts vary from hospital to hospital, but typically contain items such as the medical equipment, e.g., tools, and medication, e.g., drugs, needed to treat a person in or near immediate life danger (i.e., a cardiac arrest). These items/assets may include but are not limited to:
Defibrillators, suction devices, and Bag Valve Masks (BVMs) of different sizes;
Advanced Cardiac Life Support (ACLS) drugs;
First line drugs for treatment of common problems;
Drugs for rapid sequence intubations;
Drugs for peripheral and central venous access;
Pediatric equipment (common pediatric drugs, intubations equipment, etc.);
Medical equipment such as needles, airway devices, mask, scalpel, etc., among other medical equipment;
Other types of equipment that are capable of being stored in, on and/or attached to the cart; and
Other drugs and equipment as chosen by the facility.

Cart management relates to helping assure that a crash cart constellation, i.e., group of crash carts, and supporting processes adhere to internal and external regulations/rules. Cart management spans various business processes within an organization. These processes may range from patient support to asset and facility management, and from stock inventory to risk and compliance (quality assurance). The cart management system and processes described herein advantageously improves work efficiency, can be used to show regulatory compliancy, and have minimal impact on existing business and information technology (IT) architecture, etc. In one or more embodiments, cart management provides at least one taken from a group consisting of:
informing of actual crash cart content/items characteristics, e.g., name, type, owner, quantity, expiration dates, inspection dates, how to use, etc.;
monitoring and communicating content/item relevant usability statuses;
tracks handling of cart and items at what time;
tracks whom accessed what item and at what time;
provides event triggered automated rule based activity, action and information handling;
verification of handling of cart and items is done by authorized staff, i.e., authorized to use the system, as defined and tracked by system 10 as described herein;
allows for automated buildup of usable and accessible statistics;
provides accurate and reliable data;
is user-friendly to deploy and maintain;
increases various workflow effectiveness and efficiencies; and
is reusable and scalable.

Before describing in detail exemplary embodiments, it is noted that the embodiments reside primarily in combinations of components and processing steps related to methods, systems and management devices. Accordingly, components have been represented where appropriate by conventional symbols in the drawings, showing only those specific details that are pertinent to understanding the embodiments so as not to obscure the disclosure with details that will be readily apparent to those of ordinary skill in the art having the benefit of the description herein.

As used herein, relational terms, such as “first,” “second,” “top” and “bottom,” and the like, may be used solely to distinguish one entity or element from another entity or element without necessarily requiring or implying any physical or logical relationship or order between such entities or elements. The terminology used herein is for the purpose of describing particular embodiments only and is not intended to be limiting of the concepts described herein. As used herein, the singular forms “a”, “an” and “the” are intended to include the plural forms as well, unless the context clearly indicates otherwise. It will be further understood that the terms “comprises,” “comprising,” “includes” and/or “including” when used herein, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this disclosure belongs. It will be further understood that terms used herein should be interpreted as having a meaning that is consistent with their meaning in the context of this specification and the relevant art and will not be interpreted in an idealized or overly formal sense unless expressly so defined herein.

In embodiments described herein, the joining term, “in communication with” and the like, may be used to indicate electrical or data communication, which may be accomplished by physical contact, induction, electromagnetic radiation, radio signaling, infrared signaling or optical signaling, for example. One having ordinary skill in the art will appreciate that multiple components may interoperate and modifications and variations are possible of achieving the electrical and data communication.

Although embodiments are described herein with reference to mobile carts, such as carts used in a hospital setting, it is understood that the invention is not limited solely to these embodiments and is not limited solely to a hospital environment. It is also contemplated that the concepts and arrangements described herein can be applied to fixed storage structures, e.g., shelves, cabinets, etc., that are not mobile. It is further contemplated that the concepts and arrangements described herein can be applied to environments other than hospitals and medical facilities, e.g., automobile repair shops, factories, warehouses, etc.

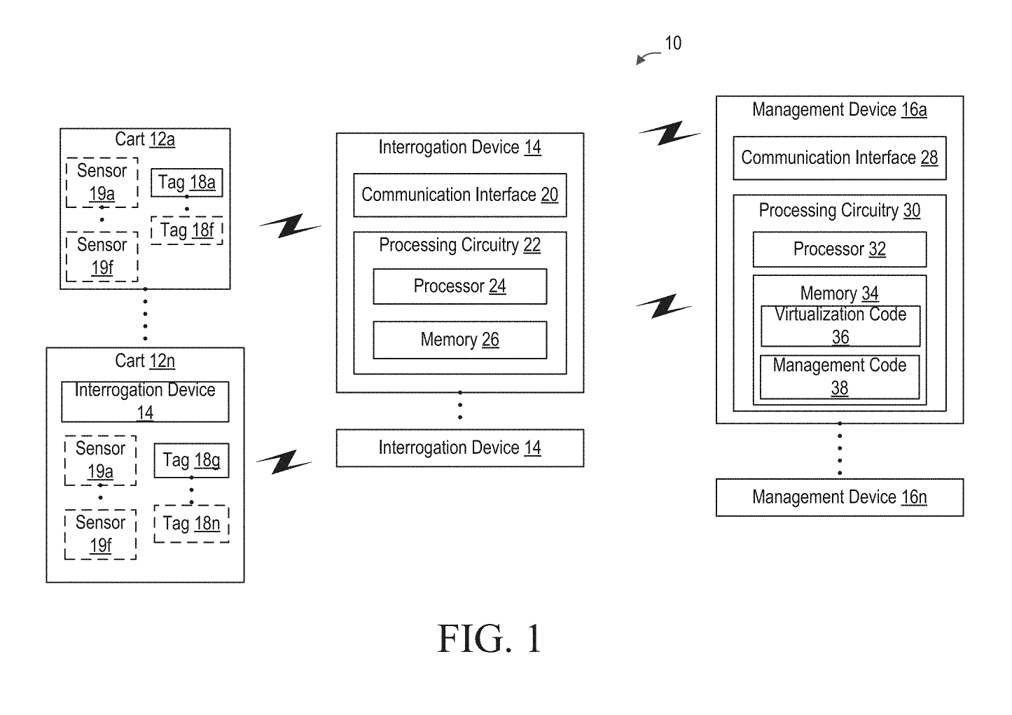

Referring now to drawing figures in which like reference designators refer to like elements there is shown in FIG. 1 is a block diagram of an exemplary system for cart compliance and inventory management in accordance with the principles of the invention. The system is generally referred to as system “10.” System 10 includes one or more carts 12a-12n, one or more interrogation devices 14a-14n and one or more management devices 16a-16n. As used herein, cart 12 refers to one or more of carts 12a-12n, interrogation device 14 refers to one or more interrogation devices 14a-14n, and management device 16 refers to one or more management devices 16a-16n.

In one or more embodiments, one or more carts 12 include one or more tags 18a-18n. As used herein, tag 18 refers to one or more of tags 18a-18n. In one or more embodiments, tag 18 includes passive, active and/or passive-active tags such as radio frequency identification (RFID) tags, BLUETOOTH low energy (BLE) tags, near field communication (NFC) tags, quick response (QR) code tag and other tags that are capable of being read by and/or communicating with interrogation device 14. In one or more embodiments, each tag 18 is associated with a specific item/component that may be at least temporarily stored or secured by cart 12. For example, medical equipment such as a nasal trumpet is associated with a specific tag 18 and is located in or on a specific cart 12. The tag 18 may be physically applied to the item. The item may include any physical item located within, on or contained by cart 12. In one or more embodiments, cart 12 includes one or more interrogation devices 14 for reading and/or communicating with tag 18. For example, individual drawers, compartments or shelves of cart 12 may have respective interrogation devices 14 for interrogating individual drawers, compartments or shelves of cart 12, thereby allowing for localized interrogation. In one or more embodiments, interrogation device 14 includes one or more of three different types of interrogation devices such as: (1) one or more cart interrogation devices 14 on cart 12; (2) one or more sub-interrogation devices 14 in communication with cart interrogation device 14 where the sub-interrogation devices 14 register/detect drawer openings and closings; and (3) a central interrogation device or reader (grid) that collects communication/data from cart interrogation devices 14 and transmits the collected communication/data to management device 16, i.e., to the back-end device(s).

In one or more embodiments, cart interrogation device 14 communicates with management device 16 via WiFi or BLE in case of sub-interrogation device 14 present in/on cart 12, or via 3G/4G cellular if sub-interrogation device 14 is not present. In one or more embodiments, cart interrogation device 14 communicates with central interrogation device 14 via BLE. In one or more embodiments, sub-interrogation device 14 communicates with tagged items through RFID or BLE with cart interrogation device 14. In one or more embodiments, any BLE enabled device can communicate with central interrogation device 14. In one or more embodiments, central interrogation device 14 communicates with cart interrogation device 14 and management device 16 via WiFi and/or BLE. In one or more embodiments, functionality of one or more of cart interrogation device 14, sub-interrogation device 14 and central interrogation device 14 is incorporated into a single interrogation device 14 located in or on cart 12.

In one or more embodiments, a read, capture and/or communication range of interrogation device 14 is configured to read tags within an interrogation zone that includes cart 12 with limited or no read range beyond the interrogation zone around cart 12 such as to help limit reading tags associated with other carts 12. Interrogation device 14 is described in further detail below.

In one or more embodiments, cart 12 includes one or more sensors 19a-19f. As used herein, sensor 19 refers to one or more of sensors 19a-19f. In one or more embodiments, sensor 19 is affixed or removably affixed to an item or cart 12 such as to detect, read and/or register one or more monitoring characteristics. In one or more embodiments, one or more monitoring characteristics includes temperature (e.g., item temperature, cart temperature, room temperature, etc.), sounds, movement, light, acceleration etc. In one or more embodiments, sensor 19 includes a tampering sensor 19 that is configured to detect tampering of an item, cart and/or other entity associate with cart 12. For example, in one or more embodiments, tampering sensor 19 is configured to indicate to interrogation device 14 that a tampering threshold has been met or that a sensor characteristic has changed (e.g., circuit has been opened), etc.

In one or more embodiments, cart 12 is a “crash” cart or code cart that includes one or more trays, one or more drawers, one or more shelves and/or one or more storage compartments, and is used in hospitals for transportation and dispensing of emergency items such as medication/equipment at the site of medical/surgical emergency for life support protocols. Cart 12, e.g., crash cart, store various items, such as medical supplies that have been tagged with respective tag 18. However, as noted above, the teachings of this invention are equally applicable to other storage device systems where inventory management is desired.

Interrogation device 14 includes one or more communication interfaces 20 that are configured to communicate with one or more tags 18 and one or more management devices 16, among other entities in system 10 via one or more communication protocols. In one or more embodiments, communication interface 20 includes one or more transmitters, receivers and/or transceivers such as an RFID transceiver, NFC transceiver, etc. In one or more embodiments, interrogation device 14 includes a capture element such as a camera and/or video camera for capturing an image of tag 18 such as an image of a QR code of tag 18 or other type of tag 18.

Interrogation device 14 includes processing circuitry 22. Processing circuitry 22 includes processor 24 and memory 26. In addition to a traditional processor and memory, processing circuitry 22 may comprise integrated circuitry for processing and/or control, e.g., one or more processors and/or processor cores and/or FPGAs (Field Programmable Gate Array) and/or ASICs (Application Specific Integrated Circuitry). Processor 24 may be configured to access (e.g., write to and/or reading from) memory 26, which may comprise any kind of volatile and/or nonvolatile memory, e.g., cache and/or buffer memory and/or RAM (Random Access Memory) and/or ROM (Read-Only Memory) and/or optical memory and/or EPROM (Erasable Programmable Read-Only Memory). Such memory 26 may be configured to store code executable by processor 24 and/or other data, e.g., data pertaining to communication.

Processing circuitry 22 may be configured to control any of the methods and/or processes described herein and/or to cause such methods, signaling and/or processes to be performed, e.g., by interrogation device 14. Processor 24 corresponds to one or more processors 24 for performing interrogation device 14 functions described herein. Interrogation device 14 includes memory 26 that is configured to store data, programmatic software code and/or other information described herein.

System 10 includes management device 16 for processing cart data received from one or more interrogation devices 14. Management device 16 includes communication interface 28 that is configured to communicate with interrogation device 14, other management devices 16 and/or other entities in system 10 via one or more communication protocols. In one or more embodiments, communication interface 28 includes one or more transmitters and one or more receivers for transmitting and receiving data from interrogation device 14. Management device 16 includes processing circuitry 30.

Processing circuitry 30 includes processor 32 and memory 34. In addition to a traditional processor and memory, processing circuitry 30 may comprise integrated circuitry for processing and/or control, e.g., one or more processors and/or processor cores and/or FPGAs (Field Programmable Gate Array) and/or ASICs (Application Specific Integrated Circuitry). Processor 32 may be configured to access (e.g., write to and/or reading from) memory 34, which may comprise any kind of volatile and/or nonvolatile memory, e.g., cache and/or buffer memory and/or RAM (Random Access Memory) and/or ROM (Read-Only Memory) and/or optical memory and/or EPROM (Erasable Programmable Read-Only Memory). Such memory 34 may be configured to store code executable by processor 32 and/or other data, e.g., data pertaining to communication, e.g., configuration and/or address data, etc.

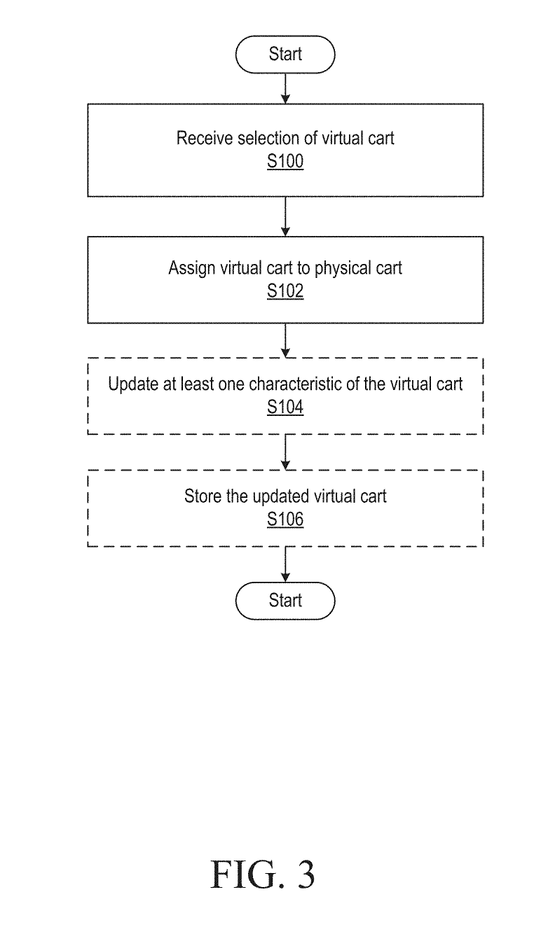

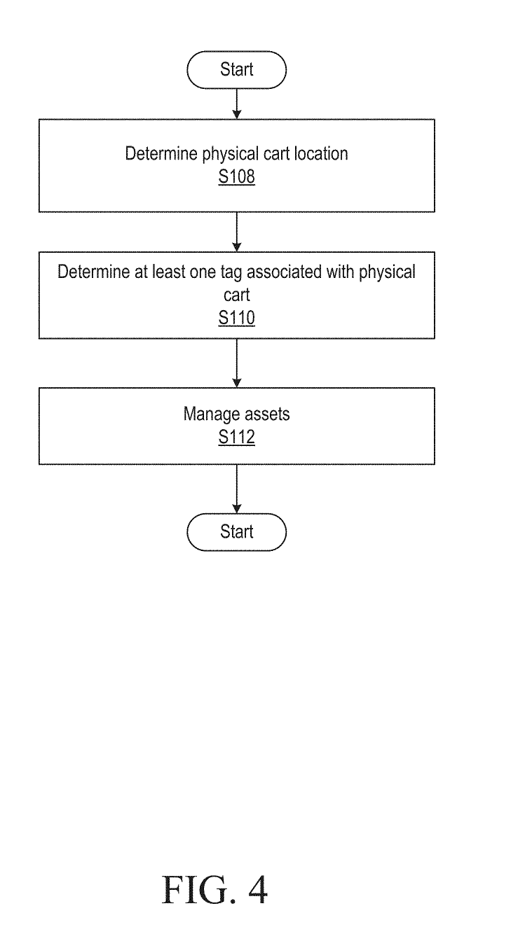

Processing circuitry 30 may be configured to control any of the methods and/or processes described herein and/or to cause such methods, signaling and/or processes to be performed, e.g., by management device 16. Processor 32 corresponds to one or more processors 32 for performing management device 16 functions described herein. Management device 16 includes memory 34 that is configured to store data, programmatic software code and/or other information described herein. In one or more embodiments, memory 34 is configured to store virtualization code 36. For example, virtualization code 36 includes instructions that, when executed by processor 32, causes processor 32 to perform the functions described herein such as the functions described with respect to FIG. 3. In one or more embodiments, memory 34 is configured to store management code 38. For example, management code 38 includes instructions that, when executed by processor 32, causes processor 32 to perform the functions described herein such as the functions described with respect to FIG. 4.

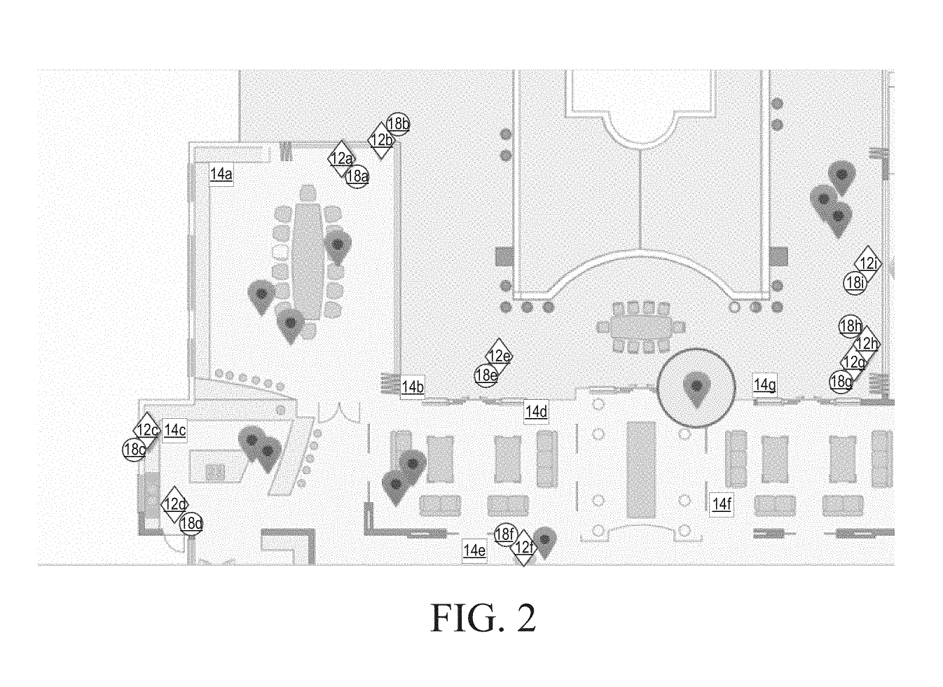

FIG. 2 is a diagram of an example of an implementation of system 10 where various interrogation devices 14a-g are positioned at various locations throughout a premises, e.g., hospital. Interrogation device 14 is configured to interrogate, via wireless signals and/or image capture, one or more tags 18a-18i associated with carts 12a-i that are located throughout the premises. In one or more embodiments, one or more tags 18a-18i are associated with people and/or other entities within the premises such as to allows for tracking of these people/entities as illustrated in FIG. 2, where the upside down teardrop symbols corresponds to tags 18a-18i associated with respective peoples and/or entities. In one or more embodiments, FIG. 2 is an example interface or screen display for the asset management system showing the location of people, e.g., medical personnel, items such as equipment, carts, etc. For example, the interface may illustrate the location of one or more carts 12, location of one or more items within cart 12, information of one or more items within cart 12 (e.g., the average cart 12 may contain 285 items where scrolling through a list of items makes inventory manageable), illustrate if an item is removed from the cart allowing automated signaling that a cart is up for a specific item replenishment, illustrate other information of items such as asset/product details, person or people responsible for item maintenance, etc.

FIG. 3 is a flow diagram of an example virtualization process of virtualization code 36 in accordance with the principles of the invention. The virtualization process advantageously creates, modifies and/or uses one or more virtual carts as database objects that can be assigned to correspond to one or more physical carts in order to track location and items associated with each physical cart according to one or more rules of the asset management process described below. Processing circuitry 30 is configured to receive a selection of a virtual cart (Block S100). For example, management device 16 receives a selection of a model of a physical cart, i.e., virtual cart. The virtual cart may have a predefined number of compartments, drawers, trays, etc. for storing or accepting one or more items associated with one or more item characteristics such as a threshold amount of an item, threshold weight of an item, concentration of an item, etc., in which the virtual cart can be modified via an interactive interface as described herein. In one embodiment, a virtual cart for an emergency room corresponds to a predefined number of compartments, drawers, trays, etc. for storing or accepting a minimum number of predefined items, i.e., a minimum number of each item or threshold amount is predefined for the virtual cart and can vary based on the type of cart 12 (e.g., emergency room type, non-emergency room type, stand-by type, etc. may each have different thresholds values associated with respective virtual carts). In another embodiment, a virtual cart for another room, i.e., non-emergency room, of the hospital corresponds to a predefined number of compartments, drawers, trays, etc. for storing or accepting a minimum number of predefined items (i.e., threshold number or amounts), one or more of which may be different from the predefined items associated with the virtual cart for the emergency room. Further, in one or more embodiments, the selected virtual cart is a model of a physical cart that is defined or created by a user, and/or a stored model of a physical cart that is modified by the user. In one or more embodiments, various virtual cart configurations are stored in memory 34 and/or in an external database accessible by management device 16, where these virtual cart configurations/models can be searched for by a user, as described herein.

In one or more embodiments, cart construct and assignments are stored in a database in memory 13, such as a relational database, where retrieval from relational database is based on authorization and organization structure assignment. In one example, staff members, i.e., users, are assigned predefined authorizations within an organizational structure where assets such as cart 12 hold the organizational structure assignment. In one or more embodiments, authorized staff members are allowed to create a virtual cart configuration model. In one or more embodiments, authorized users, such as staff members, are allowed to modify an instance based virtual cart configuration model. In one or more embodiments, system 10 allows for cart item handling of the created virtual cart instance. In one or more embodiments, system 10 allows for viewing and searching of cart 12 and cart items of the created virtual cart instance. Depending on the authorization, an assigned user, such as a staff, member logs in, navigates to the appropriate menu to look up cart 12 by, for example, description, number or assigned organizational unit, and selects a cart by querying the relational database. The relational database configuration and content are translated in a graphical user interface (GUI) representation and opens in detail for handling along the various embodiments describes herein.

Processing circuitry 30 is configured to associate a virtual cart make and model or configuration to a physical cart such as by assigning a virtual cart identifier to the physical cart (Block S102). Processing circuitry 30 assigns or associates a physical cart, i.e., cart 12, with the selected virtual cart model. In one or more embodiments, cart 12 includes tag 18 that is specific to the physical cart and used to identify cart 12 such that the virtual model is assigned to the respective tag 18 of the physical cart. In one or more embodiments, the same virtual cart is copied and assigned to multiple physical carts in which each virtual cart is assigned a different cart identifier. For example, virtual cart configuration with identifier “emergency cart” is assigned to multiple physical carts where the identifier for the assigned virtual carts are changed to “emergency cart-1”, “emergency cart-2”, etc., such that each physical cart is assigned a respective virtual cart with respective identifier, thereby allowing for ease of use in assigning virtual carts to physical carts, where each virtual cart configuration can still be modified as described herein. In one embodiment, cart 12 location is registered as physical cart 12 location via tag 18, i.e., main cart tag 18.

In one or more embodiments, a virtual cart model (template) of cart 12 is created depicting one or more of drawers, compartments as items to go on and in cart 12. Cart 12 is identified by model type, product code and/or manufacturer, etc. Various different virtual cart 12 templates can be stored in a configuration library in memory 34. Subsequently, a physical cart 12 is selected by a user and of the templates from the library is assigned to the physical cart 12, as described herein. Once the template has been assigned to physical cart 12, the virtualized cart 12 is ready for operational use. Inventory management is performed where physical items are assigned to various item templates such that virtual cart 12 representation shows which items are supposed to in physical cart 12 and which are actually in physical cart 12. As users use physical cart 12, system 10 performs item management as described herein.

Processing circuitry 30 is configured to update at least one characteristic of the virtual cart such as the predefined setting (Block S104). For example, in one or more embodiments, the characteristic of the virtual cart includes a quantity, parameters including form factors, expiration dates, supplier information, etc. For example, in one or more embodiments, one or more characteristics of the virtual cart may be modified by a user via an interface where the characteristic such a minimum number of items and/or locations of each item are stored in a database corresponding to the virtual cart. In one or more embodiments, the characteristic for a virtual cart include various minimum quantities of items and the expected locations, i.e., virtual locations, of these items in the virtual cart such that the updated virtual cart can be compared to the results of the interrogation of the physical cart for asset management as described below.

In one or more embodiments, tracking of items in/on physical cart 12 is performed by applying software application logic. For example, in one embodiment, an assumption is made that a specific drawer of physical cart 12 along the template definition of that drawer (i.e., all items belonging in the drawer are defined by a template through the configuration model). For example, an assignment assumption is that an item should be in the drawer it is assigned to. This assumption may be applied to all items that are not tagged individually by tag 18.

In the one or more embodiments incorporating the various interrogation devices, i.e., cart interrogation device 14, sub-interrogation device 14 and central interrogation device 14, discussed above, the three interrogation devices 14 are used for item tracking. Cart interrogation device 14 is linked to and put on or nearby a specific physical cart 12. Cart interrogation device 14 reads tags 18 associated with physical cart 12. Cart interrogation device 14 provides a virtual fence around the cart ranging, for example, from 1-3 meters or more. Management device 16 registers in which drawer of physical cart 12 tag 18 is placed. For example, model says item A is to be in tray one, where tag 18 associated with item A is detected within the virtual fence of physical cart 12. A user may confirm the detection such as a first detection of tag 18. Sub-interrogation device 14 registers when drawer is closed and informs cart interrogation device 14 and/or central interrogation device 14 of the closed drawer, wherein the information from tag 18 is treated as hidden. Drawer of physical cart 12 is opened and the item taken out. Sub-interrogation device 14 again registers that the tagged item is leaving, where metal confinement created by the metal physical cart 12 helps to confine interrogation zones, e.g., tag 18 may not be able to be read by cart interrogation device 14 while inside a metal drawer of cart 12 but can be read when the item is removed from the metal drawer. In other words, in one or more embodiments, interrogation zones are defined by one or more interrogation devices 14 and also by the characteristics of physical cart 12.

When tag 18 is removed from drawer of physical cart 12, cart interrogation device detects the item with tag 18. When this item with associated tag 18 leaves the set perimeter, i.e., virtual fence around physical cart 12, and is not detected for a certain period of time or returned, the assumption is made by interrogation device 14 and/or management device 16 that the item has been used or displaced. Subsequently, this event may be notified to management device 16 for further action.

Processing circuitry 30 is configured to store the updated virtual cart (Block S106). For example, processing circuitry 30 stores the updated number, locations and/or amounts of items/assets in a database. In one or more embodiments, Blocks S104 and S106 are skipped or omitted if the predefined virtual cart is not modified as the unmodified virtual cart defines the various minimum number of items for the cart and/or their various locations in the virtual cart, i.e., expected location in the physical cart.

FIG. 4 is a flow diagram of an example management process of management code 38 in accordance with the principles of the invention. Processing circuitry 30 is configured to determine a cart 12, i.e., physical cart, location (Block S108). For example, processing circuitry 30 determines the location of cart 12 such as based on a cart data provided by interrogation device 14 or based on information received from a location tracking system associated with the premises such as a geo-fencing system.

Processing circuitry 30 is configured to determine at least one tag 18 associated with a physical cart, i.e., cart 12 (Block S110). For example, processing circuitry 30 is configured to receive cart data for cart 12 in which processing circuitry 30 inventories items with tags 18 such as passive tags 18 and/or active tags 18 detected within and/or on cart 12. In one or more embodiments, cart data includes at least one of received tag identifications, one or more characteristics of one or more items, etc. In one or more embodiments, processing circuitry 30 causes interrogation device 14 proximate or associated with cart 12 to interrogate cart 12 for items. The detected items are recorded such that the quantity of each item is associated with cart 12. In one or more embodiments, each tag 18 is associated with a respective identification number such that the same identification numbers are not used by more than one tag 18. In one or more embodiments, processing circuitry 30 causes interrogation device 14 proximate or associated with cart 12 to interrogate cart 12 for items/assets. The detected items/assets are recorded in memory 34 and/or associated database such that the quantity of each item is associated with cart 12. Therefore, in one or more embodiments, tag data can identify individual items to allows for item level management.

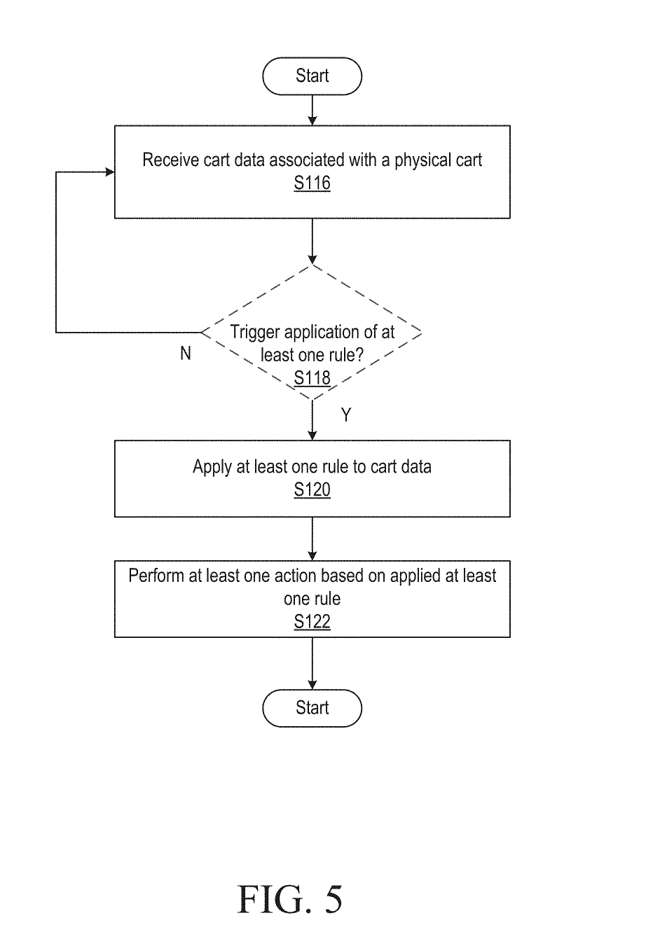

Processing circuitry 30 is configured to manage assets, i.e., items, of one or more carts 12 (Block S112). Once a cart is operationalized, items and item characteristics can be added, changed or deleted based on practical need and situation, i.e., things like the number of items available in a cart, lot numbers, expiration dates, who is responsible for maintaining what, etc., can be modified and/or stored. Items can be actively or passively tagged, enabling item level identification, monitoring and event triggering, as described herein. Block S112 is described in detail with respect to FIG. 5.

FIG. 5 is a flow diagram of an example asset management process of Block S112 in accordance with the principles of the invention. Processing circuitry 30 is configured to receive cart data associated with a physical cart, i.e., cart 12 (Block S116). The items on cart 12 will generate various data feeds that can be utilized to generate actions, support activities, build information and deliver management insights, as described herein. In one or more embodiments, each data feed from the cart 12 means data transmitted from an individual tag 18 such that each tag 18 generates a data feed. In one or more embodiments, cart 12, i.e., physical cart, interrogates tags 18 stored at cart 12 using one or more interrogation devices 14, and transmits the cart data from the interrogation to the management device 16 for processing, thereby providing one or more data feeds to management device 16. The cart data allows for cart and item location tracking, searching where items are located within cart 12 such as based on which interrogation device 14 of cart 12 received tag data associated with the item, checking stock or location of supplies, and finding where a specific cart 12 or items are located on interactive map illustrated in FIG. 2.

Processing circuitry 30 is configured to determine whether to trigger application of at least one rule (Block S118). In one or more embodiments, at least one rule is applied to the cart data in response to one or more actions, i.e., triggering actions. For example, a triggering action is at least one taken from a group consisting of detection of an open drawer of cart 12 such as by using drawer sensors included in physical cart 12 that detect opening/closing of one or more drawers, detection of cart 12 in a predefined area or zone, detection of a change of location of cart 12, detection of a change of predefined area of cart 12, a triggering due to at least one predefined time/schedule, response to receiving notification of a predefined situations occurring or expected to occur at a premises, among other triggering actions described herein.

If processing circuitry 30 determines not to trigger application of at least one rule, processing circuitry 30 performs Block S116. If processing circuitry 30 determines to trigger the application of at least one rule to cart data, processing circuitry 30 is configured to apply the at least one rule to cart data (Block S120). The one or more rules define one or more characteristics of one or more items to be housed or associated with cart 12. The one or more characteristics includes one or more of an amount of an item, a number of items, expiration date of an item and/or other quantifiable characteristics of an item.

In one or more embodiments, one or more rules are based on a location of cart 12. For example, within a first predefined zone or location, a first rule defines a minimum number of items where one or more locations within a premises correspond to one or more minimum number of items for cart 12. In a second predefined zone or location, a second rule defines a minimum number of items for cart 12, e.g., minimum of one face mask, minimum of two bottles of sodium chloride 0.9% 20 ml, etc. In other words, in one or more embodiments, different locations or zones within the premises correspond to different rules being applied, thereby allowing for cart content customization based on location (which could correspond to expected use for the cart 12 and the cart contents).

In one or more embodiments, one or more rules are based on notification of a predefined situation or emergency. In one or more embodiments, if management device 16 receives a notification or alert of a first predefined situation that is occurring or is expected to occur at the premises, processing circuitry 30 applied one or more rules that correspond to the first predefined situation. For example, a notification of a change in a predefined state, e.g., emergency, such as an allergic reaction corresponds to a third rule being applied where the third rule defines a minimum number of epinephrine injections housed in cart 12. In one or more embodiments, the one or more rules are default rules that define a minimum number of items to be housed in cart 12 such as to determine whether the number of items in cart 12 meets the number of items defined by the virtual cart assigned to cart 12. In one or more embodiments, the default rules can be used if one or more other rules are not applicable.

Processing circuitry 30 is configured to perform at least one resulting action based on the applied at least one rule, i.e., based on whether the at least one rule is met (Block S122). The at least one resulting action includes at least one taken from a group consisting of a notification to a person, employee or system 10, automatic ordering of one or more items for replenishment, triggering an audio and/or visual alert within the premises and/or on a display associated with cart 12, triggering interrogation of one or more other carts 12 within premises such as to find a specific item that is not stocked at another cart 12, among other actions.

In one or more embodiments, management device is accessible via a thin client (portal) or mobile application. Therefore, the invention advantageously allows for management device 16 to identify items, identify assets according to location, identify items in relation to one another, track item use, among other functions described herein.

In one or more embodiments, the at least one rule applied to the cart data is based on a location of the physical cart at the premises. In one or more embodiments, the premises includes a plurality of zones such as interrogation zone where each of the plurality of zones corresponds to a respective set of rules, each rule defining at least one characteristic of the at least one item. In one or more embodiments, the determining whether to trigger application of at least one rule to the cart data includes: determining whether the physical cart has moved to from a first zone to a second zone, where the application of the at least one rule to the cart data is based on the determination that the physical cart has moved from the first zone to the second zone, the plurality of zones including the first and second zones.

In one or more embodiments, the processing circuitry 30 is further configured to receive a notification of an alert at the premises where the at least one rule applied to the cart data corresponds to a predefined set of rules associated with the alert at the premises. In one or more embodiments, the processing circuitry 30 is further configured to determine whether the cart data meets the at least one rule, where the performing of the at least one action being based on the determination that the cart data does not meet the at least one rule, the at least one action including: searching the premises for at least one other physical cart, and if at least one other physical cart is found, interrogating the at least one other physical cart to determine whether the at least one other physical cart meets the at least one rule.

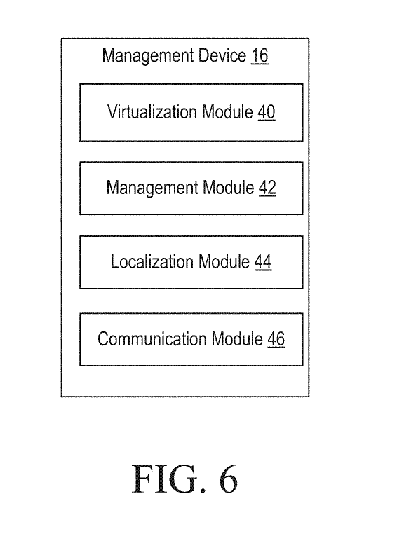

FIG. 6 is a block diagram of another example management device 16. Management device 16 includes virtualization module 40 for performing the virtualization process as described herein such as with respect to FIG. 3. Management device 16 includes management module 42 for performing the management process as describe herein such as with respect to FIGS. 4 and 5. Localization module 44 is configured to locate items, carts 12 and/or other trackable entities in system 10 such as for display on interface of FIG. 2, as described herein. Communication module 46 is configured to provide communication from management device 16 to one or more entities in system 10 as described herein.

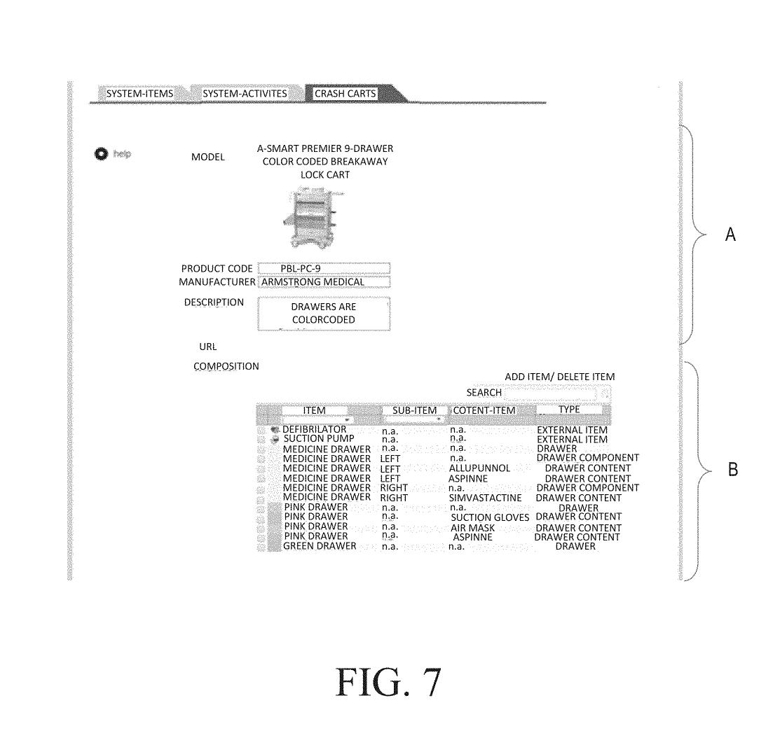

FIG. 7 is a screenshot of an example interactive interface for modifying one or more virtual cart configurations. Section A displays model information, while section B provides information about the composition or arrangement of the virtual cart configuration.

In section A, the following population fields in a database are represented:
Model (e.g., alpha numeric);
An image (e.g., to add on add image link similar as with assets);
Product code (e.g., alpha numeric);
Manufacturer (e.g., alpha numeric); and
URL (e.g., hyperlink).
   Although, other fields corresponding to one or more characteristics/compositions of the virtual cart configuration may be included. In some embodiments, the population of a field in the database may be required and, in other embodiments, the population field may be optional. The population field characteristics may be based on design and implementation requirements.

In section B, cart 12 compositions can be dynamically built and/or modified where the composition includes one or more of:
CRASH CART
   EXTERNAL ITEMS
   DRAWERS
      DRAWER CONTENT OR
      DRAWER COMPONENT
         DRAWER CONTENT
            Although, other fields corresponding to one or more compositions of the virtual cart configuration may be included.

### Some Embodiments

According to one embodiment of the invention, a management device for managing at least one item at a hospital is provided. The management device includes processing circuitry. The processing circuitry includes a memory and a processor. The processing circuitry is configured to: receive cart data associated with the at least one item of a physical cart at the hospital, determine whether to trigger application of at least one rule to the cart data, when triggered, apply the at least one rule to cart data based on the determination and perform at least one action based on the applied at least one rule.

According to one aspect of this embodiment, the at least one rule defines at least one characteristics of the at least one item. According to one aspect of this embodiment, the at least one rule is based one taken from a group consisting of: a location of the cart and a notification of a change in a predefined state. According to one aspect of this embodiment, the cart data includes at least one tag identifier associated with at least one item associated with the cart.

According to one aspect of this embodiment, the at least one action includes at least one taken from a group consisting of: generating a notification, automatically ordering at least one item, triggering a visual alert on a display associate with the cart and triggering an interrogation of at least one other cart. According to one aspect of this embodiment, the processing circuitry is configured to provide an interface for modifying a virtual cart associated with the physical cart, the virtual cart defining at least one minimum characteristic for the at least one item.

According to another embodiment of the invention, a method for a management device for managing at least one item at a hospital. Cart data associated with the at least one item of a physical cart at the hospital is received. A determination is made whether to trigger application of at least one rule to the cart data. When triggered, the at least one rule is applied to cart data based on the determination. At least one action is performed based on the applied at least one rule.

According to one embodiment of this aspect, the at least one rule defines at least one characteristics of the at least one item. According to one embodiment of this aspect, the at least one rule is based one taken from a group consisting of: a location of the cart and a notification of a change in a predefined state. According to one embodiment of this aspect, the cart data includes at least one tag identifier associated with at least one item associated with the cart.

According to one embodiment of this aspect, the at least one action includes at least one taken from a group consisting of: generating a notification, automatically ordering at least one item, triggering a visual alert on a display associate with the cart and triggering an interrogation of at least one other cart. According to one embodiment of this aspect, an interface is provided for modifying a virtual cart's construct (i.e., elements of the cart itself such as drawers within cart, items attached to drawers, etc.) and content (i.e., items to be stocked in cart) associated with the physical cart, the virtual cart defining at least one minimum characteristic for the at least one item.

As will be appreciated by one of skill in the art, the concepts described herein may be embodied as a method, data processing system, and/or computer program product. Accordingly, the concepts described herein may take the form of an entirely hardware embodiment, an entirely software embodiment or an embodiment combining software and hardware aspects all generally referred to herein as a “circuit” or “module.” Furthermore, the disclosure may take the form of a computer program product on a tangible computer usable storage medium having computer program code embodied in the medium that can be executed by a computer. Any suitable tangible computer readable medium may be utilized including hard disks, CD-ROMs, electronic storage devices, optical storage devices, or magnetic storage devices.

Some embodiments are described herein with reference to flowchart illustrations and/or block diagrams of methods, systems and computer program products. It will be understood that each block of the flowchart illustrations and/or block diagrams, and combinations of blocks in the flowchart illustrations and/or block diagrams, can be implemented by computer program instructions. These computer program instructions may be provided to a processor of a general-purpose computer (to thereby create a special purpose computer), special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable memory or storage medium that can direct a computer or other programmable data processing apparatus to function in a particular manner, such that the instructions stored in the computer readable memory produce an article of manufacture including instruction means which implement the function/act specified in the flowchart and/or block diagram block or blocks.

The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions/acts specified in the flowchart and/or block diagram block or blocks.

It is to be understood that the functions/acts noted in the blocks may occur out of the order noted in the operational illustrations. For example, two blocks shown in succession may in fact be executed substantially concurrently or the blocks may sometimes be executed in the reverse order, depending upon the functionality/acts involved. Although some of the diagrams include arrows on communication paths to show a primary direction of communication, it is to be understood that communication may occur in the opposite direction to the depicted arrows.

Computer program code for carrying out operations of the concepts described herein may be written in an object-oriented programming language such as Java® or C++. However, the computer program code for carrying out operations of the disclosure may also be written in conventional procedural programming languages, such as the “C” programming language. The program code may execute entirely on the user's computer, partly on the user's computer, as a stand-alone software package, partly on the user's computer and partly on a remote computer or entirely on the remote computer. In the latter scenario, the remote computer may be connected to the user's computer through a local area network (LAN) or a wide area network (WAN), or the connection may be made to an external computer (for example, through the Internet using an Internet Service Provider).

Many different embodiments have been disclosed herein, in connection with the above description and the drawings. It will be understood that it would be unduly repetitious and obfuscating to literally describe and illustrate every combination and subcombination of these embodiments. Accordingly, all embodiments can be combined in any way and/or combination, and the present specification, including the drawings, shall be construed to constitute a complete written description of all combinations and subcombinations of the embodiments described herein, and of the manner and process of making and using them, and shall support claims to any such combination or subcombination.

It will be appreciated by persons skilled in the art that the embodiments described herein are not limited to what has been particularly shown and described herein above. In addition, unless mention was made above to the contrary, it should be noted that all of the accompanying drawings are not to scale. A variety of modifications and variations are possible in light of the above teachings.

What is claimed is:

1. A management device for managing at least one item associated with a physical cart at a premises, the management device comprising:
processing circuitry, the processing circuitry including a memory and a processor, the processing circuitry configured to:
receive cart data associated with the physical cart at the premises;
determine whether to trigger application of at least one rule to the cart data;
when triggered, apply the at least one rule to cart data based on the determination whether to trigger application of at least one rule; and
perform at least one action based on the applied at least one rule.

2. The management device of claim 1, wherein the at least one rule applied to the cart data is based on a location of the physical cart at the premises.

3. The management device of claim 1, wherein the premises includes a plurality of zones, each of the plurality of zones corresponding to a respective set of rules, each rule defining at least one characteristic of the at least one item.

4. The management device of claim 3, wherein the determining whether to trigger application of at least one rule to the cart data includes:
determining whether the physical cart has moved to from a first zone to a second zone; and
the application of the at least one rule to the cart data being based on the determination that the physical cart has moved from the first zone to the second zone, the plurality of zones including the first and second zones.

5. The management device of claim 1, wherein the processing circuitry is further configured to receive a notification of an alert at the premises, the at least one rule applied to the cart data corresponding to a predefined set of rules associated with the alert at the premises.

6. The management device of claim 1, wherein the processing circuitry is further configured to:
determine whether the cart data meets the at least one rule;
the performing of the at least one action being based on the determination that the cart data does not meet the at least one rule, the at least one action including:
searching the premises for at least one other physical cart; and
if at least one other physical cart is found, interrogating the at least one other physical cart to determine whether the at least one other physical cart meets the at least one rule.

7. The management device of claim 1, wherein the at least one rule is taken from a group consisting of: a location of the cart and a notification of a change in a predefined state.

8. The management device of claim 1, wherein the cart data includes at least one tag identifier associated with at least one item associated with the cart.

9. The management device of claim 1, wherein the at least one action includes at least one taken from a group consisting of: generating a notification, automatically ordering at least one item, triggering a visual alert on a display associated with the cart and triggering an interrogation of at least one other cart.

10. The management device of claim 1, wherein the processing circuitry is configured to provide an interface for modifying a virtual cart associated with the physical cart, the virtual cart defining at least one minimum characteristic for the at least one item.

11. A method for a management device for managing at least one item associated with a physical cart at a premises, the method comprising:
receiving cart data associated with the physical cart at the premises;
determining whether to trigger application of at least one rule to the cart data;
when triggered, applying the at least one rule to cart data based on the determination whether to trigger application of at least one rule; and
performing at least one action based on the applied at least one rule.

12. The method of claim 11, wherein the at least one rule applied to the cart data is based on a location of the physical cart at the premises.

13. The method of claim 11, wherein the premises includes a plurality of zones, each of the plurality of zones corresponding to a respective set of rules, each rule defining at least one characteristic of the at least one item.

14. The method of claim 13, wherein the determining whether to trigger application of at least one rule to the cart data includes:
determining whether the physical cart has moved to from a first zone to a second zone; and
the application of the at least one rule to the cart data being based on the determination that the physical cart has moved from the first zone to the second zone, the plurality of zones including the first and second zones.

15. The method of claim 11, further comprising receiving a notification of an alert at the premises, the at least one rule applied to the cart data corresponding to a predefined set of rules associated with the alert at the premises.

16. The method of claim 11, further comprising determining whether the cart data meets the at least one rule; and
the performing of the at least one action being based on the determination that the cart data does not meet the at least one rule, the at least one action including:
searching the premises for at least one other physical cart; and
if at least one other physical cart is found, interrogating the at least one other physical cart to determine whether the at least one other physical cart meets the at least one rule.

17. The method of claim 11, wherein the at least one rule is taken from a group consisting of: a location of the cart and a notification of a change in a predefined state.

18. The method of claim 11, wherein the cart data includes at least one tag identifier associated with at least one item associated with the cart.

19. The method of claim 11, wherein the at least one action includes at least one taken from a group consisting of: generating a notification, automatically ordering at least one item, triggering a visual alert on a display associated with the cart and triggering an interrogation of at least one other cart.

20. A method for a management device for managing at least one item associated with a physical cart at a premises, the premises includes a plurality of zones including a first zone and a second zone, each of the plurality of zones corresponding to a respective set of rules, each rule defining at least one characteristic of at least one item, the method comprising:
receiving cart data associated with the physical cart at the premises;
determining whether to trigger application of at least one rule to the cart data, the determining whether to trigger application of at least one rule to the cart data includes determining whether the physical cart has moved to from the first zone to the second zone;
when triggered, applying the at least one rule to cart data based on the determination whether to trigger application of at least one rule;
determining whether the cart data meets the at least one rule;
performing at least one action based on the applied at least one rule, the performing of the at least one action including:
if the cart data does not meet the at least one rule, searching the premises for at least one other physical cart; and
if at least one other physical cart is found, interrogating the at least one other physical cart to determine whether the at least one other physical cart meets the at least one rule; and
generating a notification if the at least one other physical cart meets the at least one rule.
